(19) United States
(12) Patent Application Publication
(10) Pub. No.: US 2010/0179612 A1
(43) Pub. Date: Jul. 15, 2010

(54) Non-bioelectric pressure-based sensing for pacemakers and implantable cardioverting defibrillators

(76) Inventors: Michael B. Herlich, Voorhees, NJ (US); Steven D. DaTorre, Marlton, NJ (US); Michael J. Ceglia, Cherry Hill, NJ (US); S. Robert Miller, Bonita Springs, FL (US)

Correspondence Address: NORMAN E. LEHRER, ESQUIRE, 1205 NORTH KINGS HIGHWAY, CHERRY HILL, NJ, 08034, US

(21) Appl. No.: 12/657,044

(22) Filed: Jan. 13, 2010

Related U.S. Application Data
(60) Provisional application No. 61/204,959, filed on Jan. 13, 2009.

(51) Int. Cl. A61N 1/365

(52) U.S. Cl. 607/18

(57) ABSTRACT

Manually and autonomously configured non-bioelectrical-monitoring backup and/or primary pacemaker sensing is provided in the right ventricle and in the right atrium. Non-bioelectrical-monitoring is accomplished via direct, in-chamber metering and analysis of right ventricle and right atrium dynamic intracardiac pressures. A sensor is located on the right ventricular lead, another on the right atrial lead, and both are connected to the pacemaker. Right ventricular and right atrial dynamic intracardiac pressures are monitored and analyzed to indicate the presence or absence of contraction, relaxation and acceptable rhythm.

### CROSS-REFERENCE TO RELATED APPLICATIONS

This application claims the benefit of U.S. Provisional Patent Application Ser. No. 61/204,959, filed Jan. 13, 2009.

### FIELD OF THE INVENTION

This invention describes a non-bioelectrical monitoring process to prove cardiac contraction and to enhance and/or provide secure backup for an implantable pacemaker's or implantable cardioverting defibrillator's (ICD's) ability to sense (monitor) the underlying cardiac rhythm utilizing real time dynamic intracardiac pressure analysis.

### BACKGROUND OF THE INVENTION—PRIOR ART

A pacemaker is a device internally implanted to trigger the heart to beat at a normal rate should it beat too slowly on its own in a variety of pathologic processes. An ICD provides basic pacemaker functions in addition to its capability of terminating potentially lethal rapid arrhythmias. As well as the pacemaker/ICD's ability to pace the heart, it must also have the ability to accurately sense (monitor) the heart's underlying rhythm so as not to interfere with it's periodically normal function and also to accurately discriminate normal rhythm from dysrhythmia or even electrical interference. Current technology utilizes bioelectric rhythm monitoring via the same electrical conducting leads that simultaneously serve to pace the heart.

Impaired pacemaker sensing of the heart's underlying rhythm is a true clinical problem for a variety of reasons. First, finding a suitable lead placement during initial implant that allows both adequate sensing and pacing may be difficult in a variety of clinical settings. Once implanted, inflammation or scar tissue at the lead implant site may result in progressive sensing impairment. Failed sensing may lead to pacing while the heart is beating normally which results in uncomfortable palpitations or even a complex, perhaps life-threatening dysrhythmia. Electrical artifact may be incorrectly sensed as heart beats and inhibit the pacemaker from pacing, which would lead to fainting if the heartbeat was indeed slow at the time. Without proper sensing, ICD's lose the opportunity to recognize life threatening arrhythmias that require termination. Once a pacemaker loses its ability to sense correctly, the lead and/or pulse generator must be surgically removed and a new device/lead replaced for accurate function, with its attendant risk of surgical and infectious complications.

Given the necessity of accurate pacemaker/ICD sensing, it would be desirable to have alternate technology built into the leads and devices to enhance sensing or alternatively to serve as secure backup for bioelectrical sensing to avoid the life-threatening clinical complications of sensing failure and the requirement for surgical revision when electrical sensing fails.

In addition, it would be desirable to incorporate into a pacemaker or ICD the ability to be able to determine whether or not the heart is actually contracting (i.e., to prove cardiac contraction) in response to the bioelectric signals that are stimulating it to contract.

### OBJECTS AND ADVANTAGES

It is an object of this invention to describe a method for determining and utilizing non-bioelectric, cardiac hemodynamic parameters (intracardiac pressure) for pacemaker sensing and to prove cardiac contraction.

It is further an object of this invention to utilize non-bioelectric sensing for primary sensing or as a means of secure backup when primarily sensing bioelectrically.

It is further an object of this invention to enable the pacemaker/ICD to discriminate electrically sensed artifact and the true underlying heart rate.

Advantages of this invention include:

1. Alternate primary pacemaker/ICD non-bioelectric sensing capability when implanted bioelectrical sensing is suboptimal.

2. Secure backup for bioelectrical sensing.

3. Accurate discrimination between artifact and cardiac electrical activity.

4. Ability to prove cardiac contraction.

5. Reduction in surgical explant/reimplant procedures for failed electrical sensing.

### SUMMARY OF THE INVENTION

This invention accomplishes its objects by, in addition to conventional bioelectric sensing, comparing cyclical real time changes in cardiac intracardiac pressure that result from the heart's pumping action. This information is collected and inserted into algorithms that determine whether the heart should be paced or remain in monitoring mode if the heart is beating appropriately on its own.

Supplementing conventional bioelectric sensing/stimulating leads, a non-bioelectric, fluid pressure sensor is placed on one or both of the right atrial and the right ventricular leads, and each is connected electrically to the pacemaker/ICD. Cardiac pressure events are monitored by the non-bioelectric sensor(s) and used to determine or confirm when the heart requires pacing. This non-bioelectric function can be programmed as the preferred, primary means of sensing or as secure backup. As well, algorithmically perceived dysrhythmias can be addressed to discriminate between electrical artifact and real cardiac rhythm disturbance.

### BRIEF DESCRIPTION OF THE DRAWINGS

FIG. 1 representatively depicts: an implanted pacemaker/ICD controller having integral data processing means; the right atrium of the heart; and the right ventricle of the heart. A non-bioelectric, fluid pressure sensor and a conventional bioelectric sensor is depicted in each of the two chambers of the heart. An electrical lead interconnects each sensor with the controller within the conventional lead sets depicted.

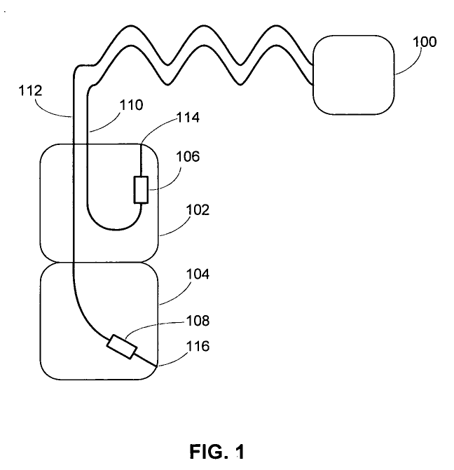

FIG. 2 comprises a flow chart of the pacemaker/ICD functions described in this invention.

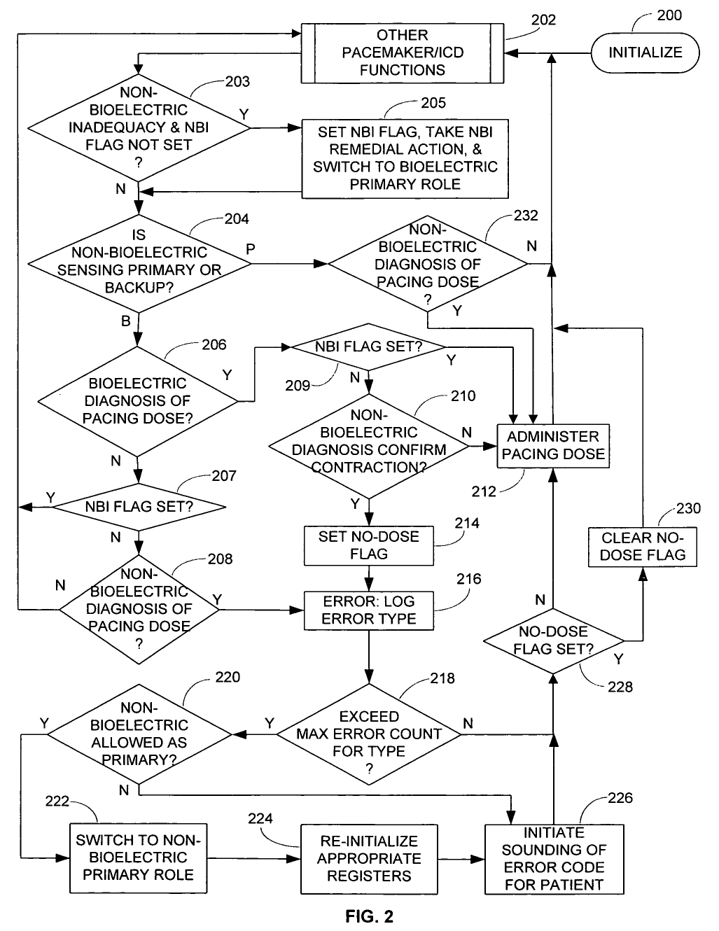

### LIST AND DESCRIPTIONS OF DRAWING ITEMS BY REFERENCE NUMBER

Item 100 depicts the patient's implanted pacemaker/ICD process controller, incorporating conventional integral data processing means, power supply, logic elements, and connections for sensing and stimulation.

Item 102 is a simplified representation of the patient's right atrium.

Item 104 is a simplified representation of the patient's right ventricle.

Item 106 represents a non-bioelectric, fluid pressure sensor in right atrium 102. Via conventional means, it is implanted and secured in right atrium 102. The design of this device is not a part of this invention. This device converts variations in intracardiac fluid pressure into equivalent electrical facsimiles, which are analyzed by process controller 100.

Item 108 represents a non-bioelectric, fluid pressure sensor in right ventricle 104. Via conventional means, it is implanted and secured in right atrium 102. The design of this device is not a part of this invention. This device converts variations in intracardiac fluid pressure into equivalent electrical facsimiles, which are analyzed by process controller 100.

Item 110 represents a conventional technology pacing/sensing lead set interconnecting sensor 106 and process controller 100.

Item 112 represents a conventional technology pacing/sensing lead set interconnecting sensor 108 and process controller 100.

Item 114 represents the conventional bioelectric sensing/stimulating tip of pacing/sensing lead set 110.

Item 116 represents the conventional bioelectric sensing/stimulating tip of pacing/sensing lead set 112.

Step 200 comprises the conventional activation and initialization of controller 100, leads unconditionally to Step 202, and includes the appropriate setting/clearing of all controller registers, flags, and memory locations to predetermined, initial states.

Step 202 comprises the performance of all other functions of controller 100. Such other functions comprise conventional operations, and are not described herein. Upon completion of Step 202, the process leads unconditionally to Step 203.

Step 203 comprises the two possible results of testing for a non-bioelectric signal that proves adequacy of cardiac contraction. If cardiac contraction proof is inadequate, the NBI (Non-Bioelectric Inadequacy) flag is tested to ensure that it has not already been set. If both conditions exist (i.e., proof of cardiac contraction is inadequate AND the NBI flag has not already been set), the process proceeds (203Y) to Step 205. If either condition does not exist (i.e., proof of cardiac contraction is adequate OR the NBI flag has already been set), the process proceeds (203N) to Step 204.

Step 204 comprises the two possible results of testing for the current configuration of controller 100. If configured for non-bioelectric sensing as the primary sensing mode (204P), the process proceeds to Step 232. If configured for bioelectric sensing as the primary sensing mode, whereby non-bioelectric sensing is the Backup mode (204B), the process proceeds to Step 206.

Step 205 comprises setting the NBI flag, implementing predetermined remedial measures, and switching operation to bioelectric sensing in the primary role.

Step 206 comprises the step of testing for a bioelectric diagnosis requiring the administration of a pacing dose to the patient, employing the primary sensing process, bioelectric sensing. If such a diagnosis is positive (206Y), the process proceeds to Step 209. If such a diagnosis is negative (206N), the process proceeds to Step 207.

Step 207 comprises testing the NBI flag to see if it has been set. If it has been set (207Y), the process loops back to Step 202. If it has not been set (207N), the process proceeds to Step 208.

In Step 208, controller 100 employs the non-bioelectric backup sensing process to confirm whether or not the patient requires administration of a pacing dose. If administration of a pacing dose is diagnosed (208Y), the bioelectric diagnosis is assumed to be in error, and the process proceeds to Step 216. If not required (208N), the bioelectric diagnosis of Step 206 is confirmed, and the process loops back to repeat Step 202.

Step 209 comprises testing the NBI flag to see if it has been set. If it has been set (209Y), the process proceeds to Step 212. If it has not been set (209N), the process proceeds to Step 210.

In Step 210, controller 100 employs the non-bioelectric backup sensing process to confirm cardiac contraction, which would negate the need for a pacing dose. If contraction is not confirmed (210N), the process proceeds to Step 212. If contraction is confirmed (210Y), the process diverts to Step 214.

Step 212 comprises the conventional step of administering a pacing dose to the patient. Upon completion of Step 212, the process unconditionally loops back to Step 202.

Step 214 sets a NO-DOSE flag, indicating that no pacing dose is to be administered. The process then proceeds unconditionally to Step 216.

In Step 216, the error determined in Step 208, or in Step 210, is logged according to its type. The process then proceeds unconditionally to Step 218.

In Step 218, controller 100 tests all logged error types to determine if the quantity of any bioelectric sensing errors has exceeded its preconfigured maximum threshold. If it has (218Y), the process proceeds to Step 220. If it has not (218N), the process proceeds to Step 228.

In Step 220, controller 100 tests to see if it has been preconfigured to allow autonomous switching to non-bioelectric sensing as its primary sensing mode. If it has (220Y), the process proceeds to Step 222. If it has not (220N), the process diverts to Step 226.

In Step 222, controller 100 switches process operation to non-bioelectric sensing as its primary sensing mode. The process then proceeds unconditionally to Step 224.

In Step 224, controller 100 re-initializes registers appropriate to the switchover implemented in Step 222. The process then proceeds unconditionally to Step 226.

Step 226 initiates an interrupt-driven background process to sound an alerting error code for the patient. Typically, this code will take the form of a predetermined series of audible beeps, and the makeup of those beeps will define the type of error encountered. The process then proceeds unconditionally to Step 228.

Step 228 comprises testing the NO-DOSE flag to see if it has been set. If it has been set (228Y), the process proceeds to Step 230. If it has not been set (228N), the process proceeds to Step 212.

Step 230 clears the NO-DOSE flag, indicating that pacing doses may be administered. The process then proceeds unconditionally to Step 202.

Step 232 comprises the step of testing for a non-bioelectric diagnosis requiring the administration of a pacing dose to the patient, employing non-bioelectric sensing as the primary sensing process. If such a diagnosis is positive (232Y), the process proceeds to Step 212. If such a diagnosis is negative (232N), the process proceeds to Step 202.

### PREFERRED EMBODIMENT OF THIS INVENTION—DETAILED DESCRIPTION

Via conventional means and methods, fluid pressure sensor 106 is inserted into right atrium 102 via electrically conducting sensing/pacing lead set 110, which is ultimately connected to implanted controller 100. Typical securing site for lead set 110 is the right atrial appendage. Similarly, fluid pressure sensor 108 is conventionally inserted into right ventricle 104 via electrically conducting sensing/pacing lead set 112, which is ultimately connected to implanted controller 100. Typical securing site for lead set 112 is the right ventricular apex.

Initially, sensors 106 and 108 are connected to a conventional external controller to determine configuration parameters for the patient's implanted pacemaker/ICD process controller 100. Employing conventional means and methods, lead sets 110 and 112 are positioned and secured to optimize pacing, and controller 100 is custom configured by the surgeon to the specific needs of the patient.

Typically via conventional interrupt driven background processes, controller 100 subsequently and continually collects, compiles, and analyzes sensor data from sensors 106, 108, 114, and 116 via pacing/sensing lead sets 110 and 112. Sensor data is sampled by controller 100 periodically, typically at 50 to 100 mSec intervals, to form mathematical facsimiles of cardiac parameters, including: intracardiac, non-bioelectric (pressure-based) cardiograms of atrial and ventricular cycles of contraction, relaxation, and rhythm; and bioelectric cardiac pacing cardiograms. Conventional parameters are applied to analyze these cardiogram data, and anomalies are flagged for controller 100′s main program to consider and act upon. For example, if a non-bioelectric-sensing diagnosis is made indicating the administration of a pacing dose, a flag specifically assigned to that condition within controller 100′s data processing means would be set. Subsequently, as in steps 208 and 232, that flag would elicit a positive response (i.e., 208Y or 232Y), and the main program would be directed accordingly.

Following process initialization Step 200, pacemaker/ICD process controller 100 performs all functions typical for an operating pacemaker/ICD (202). It next tests to determine if an error flag has been set by controller 100 (either as a part of 202 or during an interrupt process), indicating an inadequacy in the non-bioelectric monitoring signals from fluid pressure sensors 106 and/or 108 (for example, if those signals do not fall within predetermined parameters).

If such an inadequacy has been detected, and if an NBI (Non Bioelectric Inadequacy) flag has not already been set within controller 100 to indicate that the condition is existing (203Y), then (205): the NBI flag is set; predetermined action is taken to remedy the effects of the inadequacy (the details of this action are not a part of this invention); and the primary cardiac monitoring role is switched to the bioelectric mode (if not already the case). The process then (204) tests whether non-bioelectric sensing is currently cast in the primary or backup monitoring role. In 205 it was switched into the backup mode, since bioelectric monitoring was switched into the primary mode, and the process moves next to 206 via 204B. Next, controller 100 tests for a bioelectric diagnosis that requires the application of a pacing dose (206). If yes (206Y), processor 100 then tests to see if the NBI flag is set (209). If yes (209Y), processor 100 administers the required pacing dose (212), and loops back to again perform all functions typical for an operating pacemaker/ICD (202).

If non-bioelectric inadequacy had not been detected back in 203, or if the NBI flag had already been set (indicating that non-bioelectric inadequacy had been detected previously), the process advances directly (203N) to test whether non-bioelectric monitoring is in the primary or backup role (204). If it's in the primary role (204P), processor 100 next tests (232) to test for a non-bioelectric diagnosis requiring the administration of a pacing dose (the details of such diagnoses and of the administration of pacing doses are not a part of this invention). If yes (232Y), processor 100 administers the required pacing dose (212), then loops back to again perform all functions typical for an operating pacemaker/ICD (202). If not (232N), the process directly loops back to again perform all functions typical for an operating pacemaker/ICD (202).

If the NBI flag had not been detected in its set state back in 209, the process would have advanced (209N) so as to cause processor 100 to test (210) for non-bioelectric detection of acceptable contraction of the heart muscle (the details of the detection processes are not a part of this invention). If acceptable contraction is not detected (210N), processor 100 administers an appropriate pacing dose (212), then loops back to again perform all functions typical for an operating pacemaker/ICD (202). If acceptable contraction is detected (210Y), processor 100 sets its NO-DOSE flag (214) to signify that no pacing dose is to be administered, then (216) logs an error within its registers to signify that the bioelectric diagnosis of a pacing dose in 206 was in error.

Next, processor 100 tests (218) to determine if the number of errors of the type determined in 210 (via 210Y) has exceeded a predetermined count. If not (218N), processor 100 tests to see if the NO-DOSE flag has been set (228). If not (228N), processor 100 administers an appropriate pacing dose (212), then loops back to again perform all functions typical for an operating pacemaker/ICD (202). If yes (228Y), processor 100 clears its NO-DOSE flag and loops back to again perform all functions typical for an operating pacemaker/ICD (202).

If 218 reveals that the count of such errors has exceeded the predetermined limit (218Y), processor 100 tests to determine whether or not it has been preconfigured to allow non-bioelectric sensing in the primary monitoring role (220). If not (220N), processor 100 initiates an audible code within the patient (226) that defines the problem (the details of such audible codes are not a part of this invention). Processor 100 then tests the state of the NO-DOSE flag (228), and proceeds accordingly.

If processor 100 determines (220), instead, that it has been preconfigured to allow non-bioelectric sensing in the primary monitoring role (220Y), it switches non-bioelectric to the primary monitoring role (222), relegating bioelectric monitoring to the backup role. Processor 100 then re-initializes all registers appropriate to the revised monitoring roles (224), then initiates an audible code within the patient that defines the problem (226).

If, back in 206, controller 100′s test for a bioelectric diagnosis did not dictate the application of a pacing dose (206N), processor 100 next tests to see if the NBI flag is set (207). If yes (207Y), the process loops back to again perform all functions typical for an operating pacemaker/ICD (202). If not (207N), processor 100 tests for a bioelectric diagnosis that requires a pacing dose (208).

If not (208N), the process loops back to again perform all functions typical for an operating pacemaker/ICD (202). If yes (208Y), then processor 100 logs an error (216) within its registers to signify that the bioelectric diagnosis of a pacing dose in 206 was in error.

### OTHER EMBODIMENTS AND APPLICATIONS OF THE INVENTION

Essentially, only the preferred embodiment of this invention has been described. Various other embodiments, applications, and ramifications are possible within the scope of this invention. Thus, the scope of the invention should be determined by the appended claims and their legal equivalents rather than by the examples given.

### GLOSSARY

The term “configurable by the surgeon”, as used herein, is intended to refer to features of the pacemaker/controller that are configured by the surgeon at the time of implantation, and/or which may subsequently be reconfigured via conventional external controller.

The words “bioelectric” and “bioelectrical”, as used herein, are intended to refer to the natural electrical pacing signals of the heart.

The word “conventional”, as used herein, is intended to refer to means and/or methods not unique to this invention.

The acronym “ICD”, as used herein, is intended to refer to an “implantable cardioverting defibrillator”.

The term “interrupt-driven background process”, as used herein, is intended to refer to a pacemaker/controller process that is driven by event-induced data processing means interrupts, and which is not a part of the main program sequence. Such processes may take place seemingly simultaneously with the main program, and include, for example, the sending of audible error beep codes and the collection of data from the system's various sensors during the same time frame as main program execution.

The acronym “NBI”, as used herein, is intended to mean Non-Bioelectric Inadequacy, and refers to the inadequacy, for any reason, of a non-bioelectric signal to prove cardiac contraction.

The words “non-bioelectric” and “non-bioelectrical”, as used herein, are intended to refer to intracardiac pressure variations.

We claim:

1. A method for automatically providing pacing for the heart comprising:
providing an electrical pacing device and implanting the same into a person's body;
providing an electrical sensor which is adapted to sense electrical signals from the heart and which is also adapted to provide an electrical signal to the heart for stimulating the same,
implanting said electrical sensor in the right ventricle of the heart;
providing a fluid pressure transducer and implanting the same in one of the right atrium and right ventricle of the heart, said fluid pressure transducer being adapted to sense intracardiac pressures in order to confirm contractions of the heart;
monitoring the signals received from at least one of said electrical sensor and said fluid pressure transducer and providing an electrical signal to the heart to stimulate the same if said monitored signals do not meet certain predetermined criteria.

2. The method for automatically providing pacing for the heart as claimed in claim 1 further including the step of implanting a fluid pressure transducer in each of said right atrium and right ventricle of the heart.

3. The method for automatically providing pacing for the heart as claimed in claim 1 including the step of selecting one of said electrical sensor and said fluid pressure transducer as the primary sensing means and confirming the results of the signals monitored from said primary sensing means with the results of the signals monitored from the other of said sensing means as part of the decision as to whether said monitored signals do not meet said predetermined criteria.

4. The method for automatically providing pacing for the heart as claimed in claim 1 further including providing a second electrical sensor adapted to sense electrical signals from the heart and implanting said second electrical sensor in the right atrium of the heart.

5. A system for automatically providing pacing for the heart comprising an electrical pacing device adapted to be implanted into a person's body;
an electrical sensor adapted to sense electrical signals from the heart and to provide an electrical signal to the heart for stimulating the same, said electrical sensor being adapted to be implanted in the right ventricle of the heart;
a fluid pressure transducer adapted to be implanted in one of the right atrium and right ventricle of the heart, said fluid pressure transducer being adapted to sense intracardiac pressures in order to confirm contractions of the heart;
means for monitoring the signals received from at least one of said electrical sensor and said fluid pressure transducer and providing an electrical signal to the heart to stimulate the same if said monitored signals do not meet certain predetermined criteria.

6. The system for automatically providing pacing for the heart as claimed in claim 5 including a second fluid pressure transducer adapted to be implanted in the other of said right atrium and right ventricle of the heart.

7. The system for automatically providing pacing for the heart as claimed in claim 5 including means for selecting one of said electrical sensor and said fluid pressure transducer as the primary sensing means and means for confirming the results of the signals monitored from said primary sensing means with the results of the signals monitored from the other of said sensing means as part of the decision as to whether said monitored signals do not meet said predetermined criteria.

8. The system for automatically providing pacing for the heart as claimed in claim 5 further including a second electrical sensor adapted to sense electrical signals from the heart and being adapted to be implanted in the right atrium of the heart.
